(19) United States
(12) Patent Application Publication
(10) Pub. No.: US 2010/0237289 A1
(43) Pub. Date: Sep. 23, 2010

(54) Infectious waste treatment system and method

(76) Inventors: John Self, Cullman, AL (US); Robert A. Olexy, Parrish, FL (US); Steve Cochran, Baton Rouge, LA (US)

Correspondence Address: JONES, TULLAR & COOPER, P.C., P.O. BOX 2266 EADS STATION, ARLINGTON, VA, 22202, US

(21) Appl. No.: 11/488,493

(22) Filed: Jul. 18, 2006

(51) Int. Cl. B09B 3/00; A61L 2/04; C01B 3/32

(52) U.S. Cl. 252/373; 422/1; 422/107

(57) ABSTRACT

An infectious waste treatment system and method provided for treating waste material wherein the waste material is reduced and rendered decontaminated and unrecognizable thereby facilitating the disposal of infectious waste or potentially infectious waste materials. The treatment system is utilized for thermomechanically treating and processing all types of waste material. The treatment system includes a container processing unit in communication with a disinfection unit. The container processing unit is used to receive the waste materials in containerized form and convert the waste material into a usable medium for the disinfection unit. The disinfection unit comprises a system which drives a thermomechanical process wherein the waste material is rendered decontaminated and unrecognizable. The treatment system may be operated effectively without the presence of chemical disinfectants, steam, or radiation. In addition, the resulting product of the infectious waste treatment system and/or method may be used as a usable energy source in a separate process.

### BACKGROUND OF THE INVENTION

1. Field of the Invention

The present invention relates generally to the field of decontamination of waste. In particular, the present invention is directed to a method and apparatus for the thermomechanical decontamination and volume reduction of infectious waste such as medical waste.

2. Description of Related Art

In the decontamination of infectious waste or potentially infectious waste, namely medical waste, it is important to ensure that the ultimate waste product which is to be discarded is free of pathogenic microorganisms. It is also highly desirable, and in some instances required by law, to render infectious waste in a condition such that individual components, such as disposable syringes, bandages, body fluid receptacles, and even body parts removed in surgery or in autopsies, are unrecognizable and represents no continued handling hazards.

Infectious waste such as medical waste may be generated by hospitals, medical laboratories, biological laboratories, and the like and is required to be decontaminated prior to being disposed. Examples of medical waste include hypodermic syringes, glassware, slides, gauze, needles, infectious tissues, blood-soaked materials, red bag waste, or other such potentially infected or contaminated medical waste materials typically generated during the normal operation of a hospital, medical laboratory, or the like. Public concern over the proper treatment and disposal of medical waste products has increased over the past several years. This increase is due in part to an increased public awareness of the diseases that can be transmitted by biologically contaminated waste products. It is therefore desirable to produce a disposal system which adequately decontaminates and destroys infectious waste products while rendering the waste unrecognizable and minimizing the amount of contact between the medical waste products and an operator of the disposal system.

The prior art has attempted to address the problem of disposing of medical waste by methods such as specialized landfilling, incineration, steam autoclaving, chemical treatment, shredding, and/or radiation treatment. Environmental regulations have severely limited the use of incineration for infectious waste treatment due to the potential production of gaseous emissions that may contain high levels of heavy metals, volatile organics compounds, dioxins, furans, and acid gases.

Steam sterilization is another known method for treating medical waste. Steam sterilization is primarily performed in steam autoclaves. Steam autoclaving is a thermal process in which the wastes are sterilized by exposure to high-temperature steam in a pressurized environment. The high temperature and penetrability of steam are employed to destroy the infectious agents, however steam autoclaving is not efficient. For steam autoclaving to be an effective treatment method, the steam must fully penetrate the waste to ensure that all infectious microorganisms are destroyed. This full penetration by the steam may take a prolonged period of time; however, some medical waste is processed in field containers such as sharps and other dense waste which resists steam penetration. Also, since autoclaved waste is neither mechanically destroyed nor significantly reduced in volume, it is still recognizable as medical waste. Accordingly, an additional step is sometimes utilized to render the waste unrecognizable.

Chemical decontamination of infectious waste is still another method for treating infectious waste. Hospitals and other health care facilities have used chemical agents routinely for decades in the decontamination of infectious waste. As in steam autoclaving, chemical decontamination will not be effective unless there is adequate contact between the infectious waste and the chemical. In addition, the chemical will need to be maintained at a sufficient concentration and there will need to be sufficient exposure time between the waste and chemical to achieve proper levels of disinfection. Accordingly, the concentration of the chemical will have to be periodically monitored and the residence time necessary for the chemical to effectively decontaminate the waste materials may have the disadvantage of limiting throughput. There are several other disadvantages of using chemicals in the decontamination of infectious waste including potential occupational exposures of workers to chemical concentrations in the air and through skin contact; the possibility of toxic byproducts in the wastewater; chemical hazards involved with the use and storage of the chemicals; chemical residue in the treated waste; offensive odors; and no significant volume reduction of waste material.

Still another method of disinfecting infectious wastes is to use radiation treatment. The radiation may be microwave, shortwave radio, and the like. The radiation treatment suffers from several disadvantages. First, radiation treatment by itself will not render the waste unrecognizable. Second, the infectious waste must have a significant moisture content to insure effective treatment with microwaves. Third, radiation treatment will not significantly reduce the volume of the waste material.

For the foregoing reasons, there is a need for an infectious waste treatment system and method that is capable of overcoming substantially all of the short-comings of the various conventional infectious waste treatment methods while rendering the waste unrecognizable and providing a significant volume reduction of the waste.

### SUMMARY

The present invention is directed to an apparatus and method used for the thermomechanical decontamination of infectious waste that satisfies these needs [needs identified in Background section].

An infectious waste treatment system having features of the present invention comprises:
(a) an extruder comprised of:
   (i) at least one extruder screw member;
   (ii) at least one barrel wherein the barrel has an upstream end portion and a downstream end portion wherein the at least one screw member extends through the barrel;
   (iii) a receiver chamber for receiving waste material into the extruder wherein waste material is fed into the upstream end portion of the barrel;
   (iv) at least one compression chamber coupled to the receiver chamber for receiving the waste material from the receiver chamber;
   (v) an extruder outlet opening for discharge of a extrudate from the extruder located at about the downstream end portion of the barrel;
   (vi) at least one temperature sensor coupled to the extruder for measuring temperature of the waste material in the barrel;
   (vii) a motor coupled to the at least one screw member for rotating the at least one screw member; and
   (viii) a terminal control valve located at downstream end portion of the barrel for controlling temperature of the waste material in the barrel; and
(b) a process control unit coupled to the terminal control valve and to the temperature sensor for receiving a temperature signal from the temperature sensor indicative of the temperature of the waste material in the barrel wherein the position of the terminal control valve may be adjusted based on the temperature signal;
   wherein the decontamination system is utilized for rendering the waste material decontaminated and unrecognizable.

In another preferred embodiment of the infectious waste system, the extruder comprises two screw members wherein the screw members are counter interleaved counter-rotatable screws.

In another preferred embodiment of the infectious waste system, the extruder comprises at least two compression chambers in series.

A method for treating waste material having features of the present invention comprises the steps of:
introducing waste material in an extruder receiver chamber;
passing said waste material into an extruder compression chamber wherein said extruder compression chamber mixes, grinds, crushes, and compresses said waste material;
heating said waste material in extruder compression chamber;
decontaminating said waste material; and
rendering said waste material unrecognizable.

The apparatus and method of the present invention provides an efficient means to treat a variety of waste by rendering the waste decontaminated and unrecognizable. The present invention achieves this result while significantly reducing the volume of the waste.

These and other objects, advantages, and features of this invention will be apparent from the following description.

### BRIEF DESCRIPTION OF THE DRAWINGS

FIG. 1 is a schematic of a preferred embodiment of the decontamination treatment system.

FIG. 2 is a top view of a preferred embodiment of the decontamination system.

FIG. 3 is a cross sectional view of a preferred embodiment of the extruder and extruder control valves.

### DETAILED DESCRIPTION

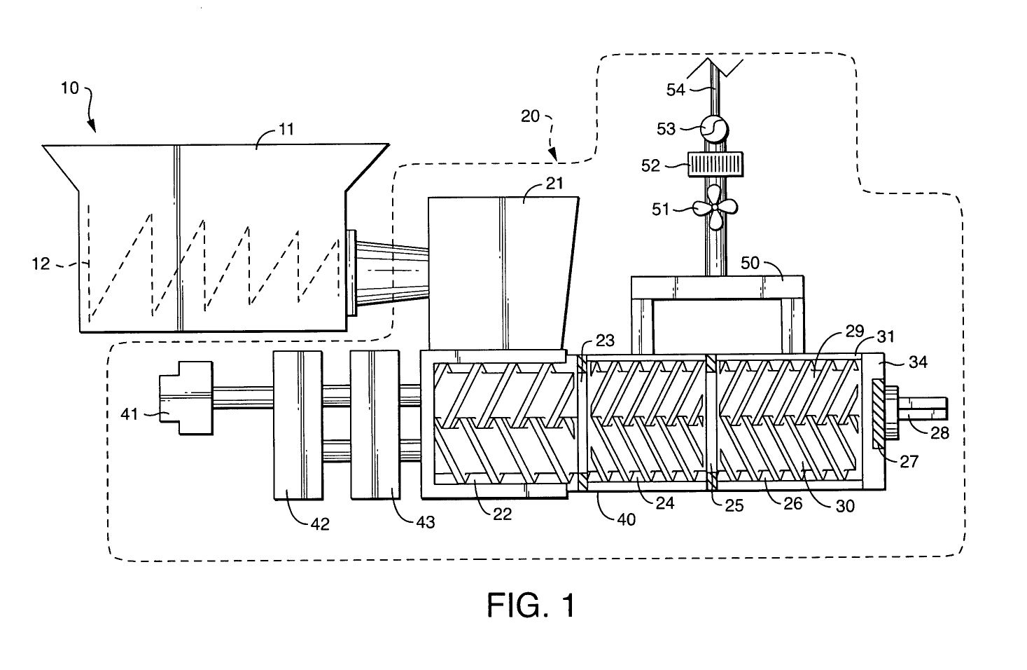

With reference to FIGS. 1-3, one or more embodiments of the infectious waste method and system will now be described. As noted above, the present invention relates to a system and method for the thermomechanical treatment of infectious waste wherein the infectious waste is rendered decontaminated and unrecognizable.

“Infectious waste” shall generally be defined as any material that is capable of either producing disease or causing infections in humans and/or animals. The definition of infectious waste shall include but shall not be limited to medical waste wherein “medical waste” is defined as any solid waste generated in the diagnosis, treatment, or immunization of human beings and/or animals, in research pertaining thereto, or in the production or testing of biologicals, excluding hazardous waste identified or listed under 40 CFR Part 261 or any household waste as defined in 40 CFR Sub-section 261.4 (b)(1). “Waste material” shall generally be defined as any material to be discarded which shall include non-infectious waste, potentially infectious waste, and infectious waste. “Decontamination” means either the substantial sterilization or disinfection of infectious waste. “Sterilization” means the removal or destruction of infectious microorganisms. “Disinfection” is a somewhat less lethal process than sterilization which destroys or inactivates viruses, fungi, and bacteria (but not necessarily their endospores) on inanimate surfaces. “Unrecognizable” means that the original appearance of the feed material has been altered such that neither the feed material nor its source can be identified. “Thermomechanical” means the combination of thermal energy and mechanical deformation.

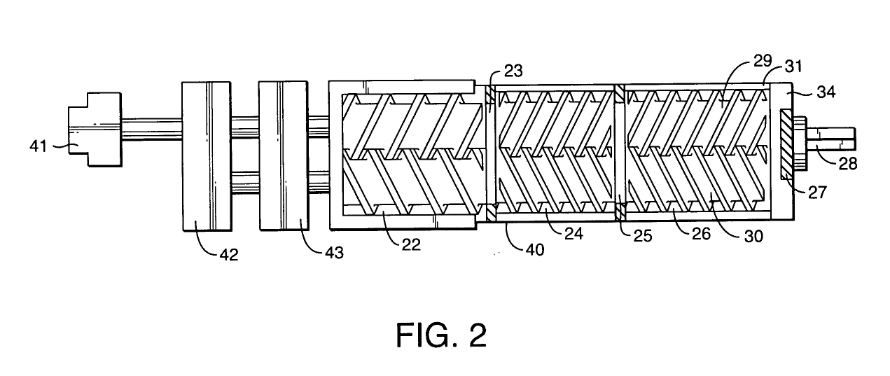

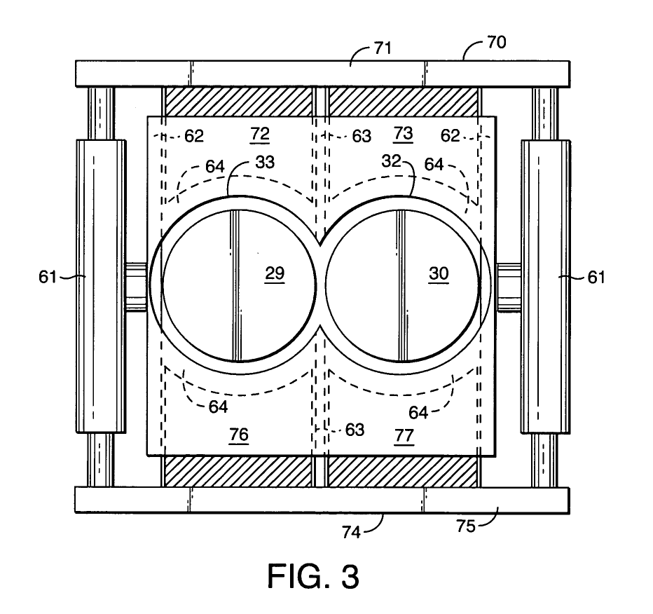

In the preferred embodiment shown in FIG. 2 and FIG. 3, the decontamination system comprises an extruder 40 wherein the extruder 40 is a screw extruder. The decontamination system is designed to treat infectious waste or potentially infectious waste that is introduced into the decontamination system whereby the waste will be decontaminated and rendered unrecognizable. The extruder 40 comprises a motor 41; a thrust bearing 42; a gear box 43; a receiver chamber 22; a first control valve 23; a first compression chamber 24; a second control valve 25; a second compression chamber 26; a terminal control valve 27; a first screw member 29, a second screw member 30; an extrusion die 28, a casing 31, an extrusion barrel 32, a material flow passage 33, and a terminal plate 34. The extrusion barrel 32 is comprised of an upstream end portion and a downstream end portion wherein the upstream end portion is near or adjacent to the receiver chamber 22 and the downstream end portion is near or adjacent to the terminal plate 34. The extruder barrel 32 is located within the first compression chamber 24 and the second compression chamber 26. The casing 31 includes all of the enclosing extruder framing and structural supports encompassing the screw members (29,30) including but not limited to the barrel 32 and terminal plate 34.

It is preferred that the extruder 40 comprise two screw members (29,30) wherein the first screw member 29 and second screw member 30 are interleaved counter-rotatable screws; however, the extruder 40 may comprise any number of screw members in any number of configurations. Furthermore, it is preferred that the screw members (29,30) be configured in a worm gear design wherein the screw member has spirally threaded flights. It is also preferred that the extruder 40 comprise two compression chambers (24,26) in series; however, extruder 40 may comprise any number of chambers so long as there is at least one compression chamber. The number of screw members and compression chambers can be adapted to the waste material to be processed and/or to the desired throughput to be achieved. It is preferred that this preferred embodiment of the infection waste treatment system be an improved modification of the extruder described in U.S. Pat. No. 4,599,002 which is directed to reducing the volume of materials. U.S. Pat. No. 4,599,002 is hereby incorporated by reference.

The extruder 40 heats, mixes, grinds, crushes, and compresses the infectious waste as the waste moves through the extruder 40. The resulting extrudate is a decontaminated material that is unrecognizable and is suitable for disposal in a landfill. Depending on the operating conditions of the extrusion process and the type of waste fed into the extruder 40, the extrudate may be substantially homogeneous in a brickqette form wherein the extrudate may be bonded and compressed so that there is volume reduction of about 95%. In addition, the resultant extrudate may be substantially free from leachate and extrudate may have diminished leachability such as when extrudate is placed in a landfill. The percentage of volume reduction may be variable depending of the type of waste, operating conditions, temperature, and rotational speed of the screw members. The amount of the mechanical energy that is supplied to the receiver chamber 22 and compression chambers (24,26) is controlled in particular by the rotational speed of the extruder screw members (29,30). The conversion of this mechanical energy into thermal energy will be dependant on the rotational speed of the extruder screw members (29,30), the operating position of the control valves (23,25,27), and the operating position of the extrusion die 28.

At least a portion of the mechanical energy of the rotating screw members (29,30) is converted into thermal energy in the extruder 40 by way of the friction imparted on the waste material by way of the rotation of the screw members wherein the waste material will grind against itself and the components of the extruder 40. Mechanical energy of motion may be converted into thermal energy when surfaces grind together, producing friction between the objects. This conversion of mechanical energy to thermal energy occurs in the extruder 40 when the waste material is compressed and grinds against other waste material and the internal components of the extruder 40. Preferably, all of the thermal energy required to decontaminate the infectious waste in the extruder 40 is introduced from this conversion of mechanical energy into thermal energy; however, there may be situations where an external source of heat is needed for a continuous operation, upon start-up of the machine, or to accommodate a special type of waste material. This external source of heat may be supplied by resistance heating, inductive heating, combustion heating, or other like sources of heat input.

The preferred embodiment of the first and second control valves (23,25) of the extruder 40 is shown in FIG. 3. Each control valve comprises an upper valve portion 70 and a lower valve portion 74. The upper valve portion 70 is comprised of a first upper plate member 72, a second upper plate member 73, and an upper cross member 71. The lower control valve portion 74 is comprised of a first lower plate member 76, a second lower plate member 77, and a lower cross member 75. Each plate member is rigidly connected to its respective cross member and has a distal end with a shape that substantially conforms to the corresponding shaft of the screw member. Each end of the cross members (71,75) is attached to the control arms 61 wherein the control arms 61 move the control valve from an open position, a closed position, and intermediate positions. In addition, each plate member (72,73,76,77) is guided by first valve guide 62 and second valve guide 63 on either side of the plate member. For example, first upper plate member 72 is guided by valve guide 62 on one side and second valve guide 63 on the other side as the first upper plate member 72 traverses from a closed position to an open position.

The control valve (23, 25) is shown in the open position in the FIG. 3 wherein the plate members (72,73,76,77) of the control valve (23,25) are recessed inside the extruder casing 31. When the control valve (23,25) is recessed into the casing 31, a void 64 is created by the absence of the plate members (72, 72, 76, 77). At each of the control valve positions, the screw members will have no flights as shown in FIG. 2, therefore the control valve can substantially engage the shaft of the screw members (29,30) when control valve is in the closed position. The shape of the distal end of the each plate member (72,73,76,77) is designed so that the valve (23,25) can substantially close off the material flow passage 33. It is understood, however, that in this preferred embodiment a small passage will exist in between the screw members (29,30) when the valve (23,25) is in the closed position. Alternatively, the valve design could be configured to eliminate this small passage between the screw members (29,30).

The terminal control valve 27 represented in FIG. 1 and FIG. 2 may be any type of valve or any mechanical devices that can control and/or prevent the flow of material from the second compression chamber into the extrusion die 28. In a preferred embodiment, the valve would be a sliding gate valve that is incorporated into the terminal plate 34 and is controlled by a hydraulic actuator. After passing through the terminal control valve, the material will then pass through an extrusion die 28. The extrusion die 28 represented in FIG. 1 and FIG. 2 may be any type of extrusion die 28 common to extruders. The extrusion die 29 is mounted at the end of the extruder 40 and gives the extrudate its final shape. It is preferred that the operating position of the extruder die 28 be controlled by a hydraulic actuator although it may be manually controlled as well.

A second preferred embodiment of the decontamination system is shown in FIG. 1. In this preferred embodiment, there are two primary components to the decontamination system. These two primary components are a feed processor unit 10 and a disinfector 20. The feed processor unit 10 is an apparatus that comprises a first feed hopper 11 and a feed mixer 12. The first feed hopper is utilized for loading and storing waste material to be treated and will feed the feed mixer 12 when the decontamination system is activated. The feed mixer 12 renders the waste material more suitable for feeding into disinfector 20 by bursting, tearing, shearing, or shredding the waste prior to being introduced to the disinfector 20. Preferably, the feed mixer 12 would comprise a screw press; however, the feed mixer 12 may comprise a shredder, hammer mill, or any other means of bursting, tearing, shearing, or shredding the waste. More preferably, the feed mixer 12 would comprise a twin screw press which will also dewater the waste prior to introducing the waste material into the disinfector 20.

The disinfector 20 comprises a second feed hopper 21; an extruder 40; an exhaust manifold 50; a fan 51; a particulate filter 52; and a condenser 53. The second feed hopper 21 is in communication with feed mixer 12 and receives waste material from the output of the feed mixer 12. The second feed hopper 21 feeds the extruder 40 wherein the second feed hopper 21 feeds the receiver chamber 22 of the extruder 40. The extruder 40 is the component of the treatment system wherein the waste is thermomechanically decontaminated and rendered unrecognizable. Vapours may be generated in the extruder 40 during the treatment process by way of evaporation wherein the resulting vapours are exported from the extruder 40 by way of an exhaust manifold 50 wherein a fan 51 evacuates the extruder 40 and the vapours are then filtered and condensed by the filter 51 and condenser 52, respectively. The exhaust manifold 50 may be located at the top and/or bottom of extruder casing 31. After the vapours pass through the condenser 52, the condensed vapour from the exhaust system will then enter into a liquid waste treatment process or other treatment device. The liquid waste treatment process may be an on-site treatment process or a municipal process.

100301 Turning now to a preferred embodiment of the method of treating infectious waste, the operation of the decontamination system will now be described by referencing FIG. 1, FIG. 2, and FIG. 3. In operation, a person will place the infectious waste into the first feed hopper 11. The infectious waste that is placed in the first feed hopper 11 generally has been placed into a disposable container or bag for convenient handling of the waste. For example, in the case of medical waste, the waste may be placed in what is known as a red bag which is a red plastic bag for disposal of certain medical waste. A level sensor (not shown) will be incorporated into the first feed hopper 11 and will activate the decontamination system when the first feed hopper has been filled to capacity. Once the system is activated, the feed mixer 12 will begin operation and will burst, shear, shred, or tear open the containers and/or bags containing the infectious waste and may also dewater the waste material depending on the type of feed mixer 12 that is utilized. The waste that is fed into the decontamination system may be infectious or potentially infectious. The waste that is potentially infectious may be treated by the decontamination system and method to provide insurance for the waste generator that the waste is rendered decontaminated even though the waste was never considered infectious.

After the infectious waste has passed through the feed mixer 12, the waste will then pass to the second feed hopper 21 which then feeds the extruder 40 by way of the receiver chamber 22. Once the feed mixer 12 is activated by the level sensor on the first feed hopper 11, the extruder 40 will also begin operation wherein the terminal control valve 27 in the extruder will close. The extruder screws (29,30) will then begin rotating at a continuous rpm level. The first control valve 23 and the second control valve 25 will then close, causing any extruded materials captured in the valve space void 64 from the last operation to be forced into the extruder barrel 32 for processing. At some point thereafter, the first control valve 23 will open causing infectious waste from the receiver chamber 22 to fill the first compression chamber 24 to be processed. Likewise, the second control valve 25 will open allowing waste material from the first compression chamber 24 to be fed into the second compression chamber 26. When the temperature of the waste material in the extruder barrel 32 reaches a pre-set temperature, the terminal control valve 27 will then open causing the treated waste material to pass through the extrusion die 28 wherein the extrudate may be in a compressed brickquette form. The temperature of the waste material may be measured at any point or multiple points either directly or indirectly along the extruder barrel. This process will ensure that all material passing through the extruder 40 will be decontaminated. The pre-set temperature may be any temperature that can render infectious waste decontaminated. The pre-set temperature range is preferably between 250° F. and 300° F. although the temperature can be pre-set to any temperature that renders the waste material decontaminated. The pre-set temperature is preferably over about 250° F., more preferably 260° F., and most preferably over 270° F.

In addition to the preferred operation of the decontamination system disclosed in the foregoing discussion, the scope of the invention can also cover alternative systems that do not utilize an extruder to thermomechanically decontaminate waste materials. These others systems may include but shall not be limited to hammermills, roller mills, cutters, pulverizers, and other equipment that has the ability to thermomechanically decontaminate the waste material or the infectious waste.

The extrudate may be collected and disposed of in a landfill or other similar location. Alternatively, the extrudate may be collected and processed in an incinerator or similar combustion processor wherein the extrudate is used as a fuel and wherein the extrudate is converted into a usable energy source. The incinerator may be located at the decontamination site or off-site in order to recover the BTU value of the extrudate. Furthermore, the extrudate may be collected and further transformed in a steam reformation process wherein the extrudate will be at least partially converted into a syngas comprising primarily of hydrogen, carbon monoxide, methane and carbon dioxide.

As mentioned in the foregoing description of the invention, the decontamination system may be operated wherein substantially all of the mechanical energy input to the rotation of the screw members is converted into thermal energy within the extruder 40. The decontamination system may also be operated without the addition of disinfectant chemicals. The temperature within the extruder 40 may drive the decontamination process without the need of these disinfecting chemicals. In addition, the decontamination system may be operated without the need of any type of radiation source such as microwaves or radio waves. Furthermore, the decontamination system may be operated without the additional of steam from an external source with the understanding that some steam may be generated within the decontamination system due to the operating temperature. The decontamination system will be preferably operated between 250° F. and 300° F. and will therefore be preferably operated in a temperature range that will prevent the incineration, combustion, and/or the thermal oxidation of the waste material. However, all of the methods disclosed can be incorporated into the decontamination system if the operator so chooses.

During steady state conditions of the decontamination process, the control valves (23,25,27) may operate at any position wherein the pre-set temperature of the waste material is achieved. In addition, the extruder die 28 is adjustable and may be adjusted to provide an extrudate to desired dimensions and specifications. When the first feed hopper 11 is empty or substantially empty, a level sensor (not shown) signals the decontamination system to go into shut-down mode at which time the decontamination system will no longer operate at steady state. When the system begins the shut-down mode, the first and second control valves (23,25) will fully open and a portion of the extruded waste material will be forced out of the extruder barrel 32 and into the valve space void 64. In addition, the terminal control valve 27 will fully open and allow the material to pass through the extrusion die 28.

On the intake side of extruder 40 adjacent to the receiver chamber 22, two drive shafts connect to screw members (29,30). The drive shafts project out of the casing 31 and extend through gear box 43 and are supported on a thrust bearing 42. The extruder motor 41 is coupled to one of the drive shafts wherein the motor 41 will rotate the corresponding screw member when in operation. The drive shaft that is being driven by the motor 41 is engaged with the other drive shaft by way of the gear box 43 wherein the rotational energy of one screw is transferred to the other thereby rotating both screw members (29,30) at the same speed in a counter rotating manner. The motor 41 is preferably a hydraulic motor.

In a preferred embodiment, a process control unit (not shown) regulates the operation of the entire decontamination system. The process control system may be a conventional, microprocessor-based process controller, a process logic controller, or similar process control system. The process control system responds to signals from the temperature sensor(s) and regulates the operation of the extruder 40. The process control system prevents the output of waste from the extruder 40 until the temperature of the extruder material flow passage is at least the required minimum temperature that is pre-set into the process control unit. The process control system regulates the first and second control valves (23, 25) and the terminal control valve 27 within the extruder 40 until the temperature within the extruder material flow passage 33 reaches the required temperature to effectively decontaminate the infectious waste. The process control unit may also control the speed of rotation of the screw members (29,30) by the regulation of the speed of the motor 41. The process control unit also monitors the start-up and shut-down of the infectious waste system by way of monitoring the first hopper level sensor and receiving a signal from the level sensor.

Insofar as the description above and the accompanying drawings disclose any additional subject matter that is not within the scope of the claims below, the inventions are not dedicated to the public and the right to file one or more applications to claim such additional inventions is reserved.

There are of course other alternate embodiments which are obvious from the foregoing descriptions of the invention, which are intended to be included within the scope of the invention, as defined by the following claims.

What is claimed is:

1. A decontamination system for thermomechanically treating waste material comprising:
(a) an extruder comprised of:
(i) at least one extruder screw member;
(ii) at least one barrel, said at least one barrel having an upstream end portion and downstream end portion, wherein said at least one screw member extends through said at least one barrel;
(iii) a receiver chamber for receiving said waste material into said extruder wherein waste material is fed into said upstream end portion of said at least one barrel;
(iv) at least one compression chamber coupled to said receiver chamber for receiving said waste material from said receiver chamber;
(v) an extruder outlet opening for discharge of a extrudate from said extruder located at about said downstream end portion of said at least one barrel;
(vi) at least one temperature sensor coupled to said extruder for measuring temperature of said waste material in said at least one barrel;
(vii) a motor coupled to said at least one screw member for rotating said at least one screw member; and
(viii) a terminal control valve located at downstream end portion of said barrel for controlling temperature of said waste material in said at least one barrel; and
(b) a process control unit coupled to said terminal control valve and to said temperature sensor for receiving a temperature signal from said temperature sensor indicative of the temperature of said waste material in said at least one barrel wherein the position of said terminal control valve may be adjusted based on said temperature signal;
wherein said decontamination system is utilized for rendering said waste material decontaminated and unrecognizable.

2. The decontamination system of claim 1 wherein said decontamination system is utilized for rendering said waste material sterile.

3. The decontamination system of claim 1 wherein said waste material is infectious waste.

4. The decontamination system of claim 1 wherein said waste material is medical waste.

5. The waste material treatment system of claim 1 wherein said extruder further comprises at least one additional screw member.

6. The decontamination system of claim 1 wherein said process control unit is further coupled to said motor and to said temperature sensor for receiving a temperature signal from said temperature sensor indicative of the temperature of said waste material in said barrel and for adjusting said motor speed.

7. The decontamination system of claim 1, said extruder further comprising:
(a) one or more additional said compression chambers wherein said compression chambers are connected in series; and
(b) at least one throughput control valve positioned between said compression chambers for adjusting the waste material throughput from one of said compression chamber to the next said compression chamber.

8. The decontamination system of claim 6 wherein said throughput control valve comprises:
(a) an upper valve portion positioned in between said compression chambers;
(b) a lower valve portion positioned in between said compression chambers; and
(c) at least one control arm coupled to said upper valve portion and said lower valve portion for adjusting said upper valve portion and said lower valve portion;
wherein said upper valve portion and lower valve portion are adjusted to control throughput through successive said compression chambers.

9. The decontamination system of claim 6 wherein said extruder further comprises at least one additional screw member.

10. A decontamination system for thermomechanically treating waste material comprising:
(a) a first feeder hopper for loading said waste material;
(b) a feed mixer in communication with first feed hopper; and
(c) a second feeder hopper wherein said second feeder hopper is in communication with feed mixer and wherein said second feeder hopper receives the output of said feed mixer;
(d) an extruder in communication with said second feed hopper, said extruder comprising:
(i) at least one extruder screw member;
(ii) at least one barrel said at least one barrel having an upstream end portion and downstream end portion wherein said at least one screw member is extending through said at least one barrel;
(iii) a receiver chamber for receiving waste material into said extruder wherein waste material is fed into said upstream end portion of said barrel;
(iv) at least one compression chamber;
(v) an outlet opening for discharge of a extrudate from said extruder located at about said downstream end portion of said barrel;
(vi) at least one temperature sensor coupled to said extruder for measuring temperature of waste material in said barrel;
(vii) a motor coupled to said at least one screw member for rotating said at least one screw member; and
(viii) a terminal control valve located at downstream end portion of said barrel for controlling temperature of waste material in said barrel;
(e) a process control unit coupled to said terminal control valve and to said temperature sensor for receiving a temperature signal from said temperature sensor indicative of the temperature of said waste material in said barrel and for adjusting said terminal control valve;
wherein said waste treatment system is utilized for rendering said waste material decontaminated and unrecognizable.

11. The decontamination system of claim 10 wherein said decontamination system is utilized for rendering said waste material sterile.

12. The decontamination system of claim 10 wherein the waste material is infectious waste.

13. The decontamination system of claim 10 wherein said waste material is medical waste.

14. The decontamination system of claim 10 wherein said process control unit is further coupled to said motor and to said temperature sensor for receiving a temperature signal from said temperature sensor indicative of the temperature of said waste material in said barrel and for adjusting said motor speed.

15. The decontamination system of claim 10 wherein said extruder further comprises an exhaust manifold coupled to said extruder for evacuating vapors generated in said extruder.

16. A decontamination system comprising:
(a) a first feeder hopper for loading waste material;
(b) a feed mixer in communication with first feed hopper; and
(c) a second feeder hopper wherein said second feeder hopper is in communication with feed mixer and wherein said second feeder hopper receives the output of said feed mixer;
(d) an extruder in communication with said second feed hopper, said extruder comprising:
(i) two extruder screw members;
(ii) a barrel said barrel having an upstream end portion and downstream end portion wherein said at least one screw member is extending through said barrel;
(iii) a receiver chamber for receiving waste material into said extruder wherein waste material is fed into said upstream end portion of said barrel;
(iv) a plurality of compression chambers positioned in series;
(v) at least one throughput control valve positioned in between said compression chambers for adjusting the waste material throughput from one of said compression chambers to the next said compression chamber.
(vi) an outlet opening located at about said downstream end portion of said barrel for discharge of extrudate from said extruder;
(vii) at least one temperature sensor coupled to said extruder for measuring temperature in said barrel;
(viii) a motor coupled to said at least one screw member for rotating said at least one screw member; and
(ix) a terminal control valve located at downstream end portion of said barrel for controlling temperature of waste material in said barrel before said waste material exits said barrel;
(x) an exhaust manifold coupled to said extruder for evacuating vapors generated in said extruder.
(e) a process control unit coupled to said terminal control valve and to said temperature sensor for receiving a temperature signal from said temperature sensor indicative of the temperature in said barrel and for adjusting the operating position of any of said control valves based on said temperature signal and for adjusting the speed of said motor based on said temperature signal;
wherein said waste treatment system is utilized for rendering said waste material decontaminated and unrecognizable.

17. A method for treating waste material comprising the steps of:
introducing said waste material in an extruder receiver chamber;
passing said waste material into an extruder compression chamber wherein said extruder compression chamber compresses, mixes, grinds, and crushes said waste material;
heating said waste material in said extruder compression chamber;
decontaminating said waste material; and
rendering said waste material unrecognizable.

18. The method of claim 17 further comprising the steps of:
measuring temperature of said waste material in said extruder compression chamber; and
adjusting a terminal control valve on said extruder based on temperature of waste material.

19. The method of claim 17 further comprising the step of feeding the extrudate of said extruder compression chamber into an incinerator wherein said extrudate is utilized as a fuel source.

20. The method of claim 17 further comprising the step of feeding the extrudate of said extruder compression chamber into a steam reforming process wherein said extrudate is substantially converted into a syngas.

21. The method of claim 17 further comprising the step of maintaining an operating temperature of at least about 250° F. within the barrel of said extruder.

22. The method of claim 17 further comprising the step of:
measuring the rotational speed of at said least one extruder screw member; and
adjusting the rotational speed of at least one extruder screw member based on said temperature in said extruder compression chamber.

23. The method of claim 22 wherein said waste material is infectious waste.

24. The method of claim 22 wherein said waste material is medical waste.

25. The method of claim 22 wherein said operating temperature is at least about 250° F.

26. The method of claim 22 wherein said operating temperature is at least about 270° F.

27. The method of claim 22 wherein no disinfectant chemicals are used in the treatment process.

28. The method of claim 22 wherein no external source of steam is used in the treatment process.

29. The method of claim 22 wherein no radiation is used in the treatment process.

30. The method of claim 22 wherein no disinfectant chemicals; external source of steam; or radiation is used in the treatment process.

31. The method of claim 22 wherein said waste material is not incinerated.

32. The method of claim 22 wherein said waste material is not substantially oxidized.

33. A method for treating infectious waste comprising the steps of:
providing an extruder which is comprised of a receiver chamber and at least one compression chamber;
providing infectious waste;
introducing said infectious waste into said receiver chamber wherein said receiver chamber introduces said infectious waste into said at least one compression chambers;
compressing infectious waste wherein the waste material is thermomechanically heated;
decontaminating said infectious waste; and
rendering said infectious waste unrecognizable.

34. A method for the thermomechanical decontamination of infectious waste comprising the steps of:
providing infectious waste;
providing a mechanical energy output;
compressing and deforming said infectious waste utilizing said mechanical energy output;
converting said mechanical energy output into thermal energy by way of said compressing and deforming;
heating said infectious waste to an effective temperature that will render the infectious waste decontaminated;
measuring the temperature of said infectious waste;
adjusting said temperature of said infectious waste based on said temperature;
decontaminating said infectious waste; and
rendering said infectious waste unrecognizable.

35. The method of claim 34 wherein said infectious waste is rendered sterile.

36. An apparatus for treating infectious waste comprising:
means for providing a mechanical energy output;
means for compressing and deforming said infectious waste utilizing said mechanical energy output;
means for converting said mechanical energy output into thermal energy by way of said compressing and deforming;
means for heating said infectious waste to an effective temperature that will render the infectious waste decontaminated;
means for measuring the temperature of said infectious waste;
means for adjusting said temperature of said infectious waste based on said temperature;
wherein said infectious waste is rendered decontaminated and unrecognizable.

37. A method for the thermomechanical treatment of waste material waste comprising the steps of:
providing said waste material;
providing a mechanical energy output;
compressing and deforming said waste material utilizing said mechanical energy output;
converting said mechanical energy output into thermal energy by way of said compressing and deforming;
heating said infectious waste to an effective temperature that will render said waste material decontaminated;
measuring the temperature of said waste material;
adjusting said temperature of said waste material based on said temperature;
decontaminating said waste material; and
rendering said waste material unrecognizable.

38. The method of claim 37 wherein said waste material is rendered sterile.

39. An apparatus for treating waste material comprising:
means for providing a mechanical energy output;
means for compressing and deforming said infectious waste utilizing said mechanical energy output;
means for converting said mechanical energy output into thermal energy by way of said compressing and deforming;
means for heating said infectious waste to an effective temperature that will render the infectious waste decontaminated;
means for measuring the temperature of said infectious waste;
means for adjusting said temperature of said infectious waste based on said temperature;
wherein said infectious waste is rendered decontaminated and unrecognizable.
